(19) United States
(12) Patent Application Publication
(10) Pub. No.: US 2025/0221688 A1
(43) Pub. Date: Jul. 10, 2025

(54) ULTRASOUND IMAGE PROCESSING METHOD, ULTRASOUND IMAGING APPARATUS, AND ULTRASOUND SYSTEM

(71) Applicant: GE Precision Healthcare LLC, Waukesha, WI (US)

(72) Inventors: Yongchao Yang, Shanghai (CN); Hao Gu, Shanghai (CN); Xueying Yu, Shanghai (CN)

(21) Appl. No.: 19/007,127

(22) Filed: Dec. 31, 2024

(30) Foreign Application Priority Data
Jan. 4, 2024 (CN) 202410013919.8

(51) Int. Cl. A61B 8/14; A61B 8/00

(52) U.S. Cl. CPC A61B 8/14; A61B 8/40

(57) ABSTRACT

An ultrasound image processing method, comprising: acquiring an ultrasound image in a real-time ultrasound scanning process, the ultrasound image being generated by processing an ultrasonic echo signal of a currently scanned site acquired by a probe; automatically determining the currently scanned site in the real-time ultrasound scanning process; automatically selecting, from a plurality of image processing tools, an image processing tool corresponding to the currently scanned site, wherein the plurality of image processing tools correspond to a plurality of scanned sites of a scanned subject, respectively; and processing the ultrasound image using the automatically selected image processing tool.

### CROSS-REFERENCE TO RELATED APPLICATION

This application claim priority to Chinese Patent Application No. 202410013919.8, which was file on Jan. 4, 2024 at the Chinese Patent Office. The entire contents of the above-listed application are incorporated by reference herein in their entirety.

### TECHNICAL FIELD

The present invention relates to the field of ultrasound imaging, and relates in particular to an ultrasound image processing method, an ultrasound imaging apparatus, and an ultrasound system.

### BACKGROUND

Ultrasound imaging technology generally uses a probe to send an ultrasonic signal to a scanned site and receive an ultrasonic echo signal. The echo signal is then processed to obtain an ultrasound image of the scanned site. On the basis of this principle, ultrasound imaging is suitable for real-time, non-destructive scanning performed on subjects to be scanned.

After the ultrasound image is acquired, further processing of the ultrasound image is usually required to meet the clinical needs of a physician. Ultrasound images of different scanned sites, even of the same scanned site of different scanned subjects, typically require different image processing. For example, image processing may include post-optimization processing of image quality, identification of regions of interest in the image (e.g., lesion identification), calculation of some parameters in the image, etc. Some auxiliary image processing tools have been developed for the image processing described above. Also, because different images require different processing, various image processing tools have been developed for the processing of ultrasound images of different scanned sites. In a conventional use scenario, a user may select a corresponding image processing tool for real-time ultrasound image processing according to a specific scanned site.

### SUMMARY

The inventors have found that there is some difficulty for users in selecting an appropriate ultrasound image processing tool in the real-time ultrasound scanning process. The reason is that the user usually needs to operate a probe with one hand to adjust the angle and position of the probe, and operate a user interface of an ultrasound imaging apparatus, such as a keyboard or a touch panel, with the other hand, to perform operations such as storage and adjustment of images. Usually, the user has no time to perform additional manual operations, such as selecting an image processing tool. If selection of an image processing tool requires stopping operation of the probe or operations such as current image storage and adjustment, this will interrupt the continuity of the user performing the ultrasound scanning, thereby causing a decrease in ultrasound scanning efficiency.

The aforementioned defects, deficiencies, and problems are solved herein, and these problems and solutions will be understood through reading and understanding the following description.

Some embodiments of the present application provide an ultrasound image processing method, comprising:
Acquiring an ultrasound image in a real-time ultrasound scanning process, the ultrasound image being generated by processing an ultrasonic echo signal of a currently scanned site acquired by a probe;
Automatically determining the currently scanned site in the real-time ultrasound scanning process;
Automatically selecting, from a plurality of image processing tools, an image processing tool corresponding to the currently scanned site, wherein the plurality of image processing tools correspond to a plurality of scanned sites of a scanned subject, respectively; and
Processing the ultrasound image using the automatically selected image processing tool.

Some embodiments of the present application provide an ultrasound imaging apparatus, comprising a probe and a processor. The probe is used to acquire an ultrasonic echo signal of a currently scanned site. The processor is configured to perform the following method: acquiring an ultrasound image in a real-time ultrasound scanning process, the ultrasound image being generated by processing an ultrasonic echo signal of a currently scanned site acquired by a probe; automatically determining the currently scanned site in the real-time ultrasound scanning process; automatically selecting, from a plurality of image processing tools, an image processing tool corresponding to the currently scanned site, wherein the plurality of image processing tools correspond to a plurality of scanned sites of a scanned subject, respectively; processing the ultrasound image using the automatically selected image processing tool.

Some embodiments of the present application provide an ultrasound system, comprising an image processing apparatus. The image processing apparatus comprises an image acquisition unit and a processing unit. The image acquisition unit is used to acquire the ultrasound image. The processing unit is configured to perform: acquiring an ultrasound image in a real-time ultrasound scanning process, the ultrasound image being generated by processing an ultrasonic echo signal of a currently scanned site acquired by a probe; automatically determining the currently scanned site in the real-time ultrasound scanning process; automatically selecting, from a plurality of image processing tools, an image processing tool corresponding to the currently scanned site, wherein the plurality of image processing tools correspond to a plurality of scanned sites of a scanned subject, respectively; processing the ultrasound image using the automatically selected image processing tool.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic diagram of an ultrasound imaging apparatus according to some embodiments of the present application;

FIG. 2 is a schematic diagram of an ultrasound system in some embodiments of the present application;

FIG. 3 is a flowchart of an ultrasound image processing method in some embodiments of the present application;

FIG. 4 is a schematic diagram of using a sensing device to determine a relative position of a probe with respect to a scanned subject in some embodiments of the present application;

FIG. 5 is a schematic diagram of using a sensing device to determine a relative position of a probe with respect to a scanned subject in some other embodiments of the present application;

FIG. 6 is a schematic diagram of a plurality of image processing tools in some embodiments of the present application; and

FIG. 7 is a flowchart of an ultrasound image processing method in some other embodiments of the present application.

### DETAILED DESCRIPTION

Specific embodiments of the present invention will be described below. It should be noted that in the specific description of the embodiments, it is impossible to describe all features of the actual embodiments of the present invention in detail, for the sake of brief description. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. “First”, “second”, and similar words used in the present invention and the claims do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

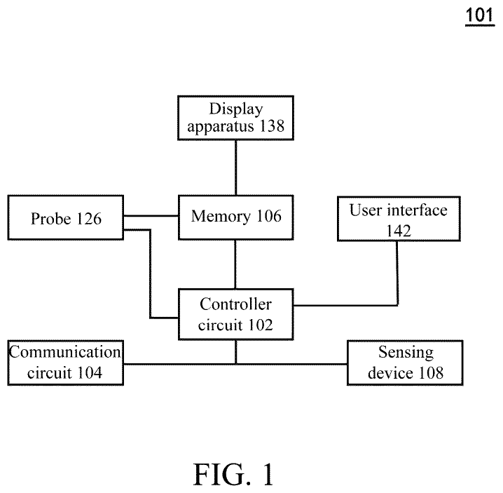

FIG. 1 shows a schematic block diagram of an embodiment of an ultrasound imaging apparatus 101. The ultrasound imaging apparatus 101 may include a controller circuit 102, a display apparatus 138, a user interface 142, a probe 126 and a memory 106, which can be operatively connected to a communication circuit 104.

The controller circuit 102 is configured to control operation of the ultrasound imaging apparatus 101. The controller circuit 102 may include one or more processors. Optionally, the controller circuit 102 may include a central processing unit (CPU), one or more microprocessors, a graphics processing unit (GPU), or any other electronic component capable of processing inputted data according to a specific logic instruction. Optionally, the controller circuit 102 may include and/or represent one or more hardware circuits or circuit systems, and the hardware circuit or circuit system includes, is connected to, or includes and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more alternate ultrasound imaging systems, remote servers, etc. The remote server may represent patient information, a machine learning algorithm, a remotely stored medical image from a previous scan, and/or a diagnosis and treatment period of a patient, etc. The communication circuit 104 may represent hardware for transmitting and/or receiving data along a bidirectional communication link. The communication circuit 104 may include a transceiver, a receiver, a transceiver, etc., and an associated circuit system (e.g., an antenna) used for communication (e.g., transmitting and/or receiving) in a wired and/or wireless manner with one or more alternative external systems, remote servers, etc. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternative ultrasound imaging systems, remote servers, etc. The bidirectional communication link may be based on a standard communication protocol, such as Ethernet, TCP/IP, Wi-Fi, 802.11, a customized communication protocol, Bluetooth, etc.

The controller circuit 102 is operatively connected to the display apparatus 138 and the user interface 142. The display apparatus 138 may include one or more liquid crystal display apparatuses (e.g., light emitting diode (LED) backlights), organic light emitting diode (OLED) display apparatuses, plasma display apparatuses, CRT display apparatuses, and the like. The display apparatus 138 may display patient information, one or more medical images and/or videos, a graphical user interface, or a component received by the display apparatus 138 from the controller circuit 102, one or more 2D, 3D, or 4D ultrasound image data sets from ultrasound data stored in the memory 106, or anatomical measurement, diagnosis, processing information, and the like currently acquired in real time.

The user interface 142 controls the operation of the controller circuit 102 and the ultrasound imaging apparatus 101. The user interface 142 is configured to receive an input from a clinician and/or an operator of the ultrasound imaging apparatus 101. The user interface 142 may include a keyboard, a mouse, a trackball, a touch pad, one or more physical buttons, and the like. Optionally, the display apparatus 138 may be a touch screen display apparatus that includes at least a part of the user interface 142. For example, a part of the user interface 142 may correspond to a graphical user interface (GUI) that is generated by the controller circuit 102 and is shown on the display apparatus 138. The touch screen display apparatus may detect the presence of a touch from the operator on the display apparatus 138, and may also identify the location of the touch relative to the surface area of the display apparatus 138. For example, a user may select, by touching or contacting the display apparatus 138, one or more user interface components of the user interface (GUI) shown on the display apparatus. User interface components may correspond to icons, text boxes, menu bars, etc., shown on the display apparatus 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include one or more learning algorithms (e.g., deep learning algorithms including a convolutional neural network algorithm, machine learning algorithms such as a decision tree learning algorithm, a conventional computer vision algorithm, or the like) configured to define an image analysis algorithm. These algorithms may form a plurality of image processing tools, and corresponding image processing tools may be selected according to different currently scanned sites of the scanned subject. The configuration of the plurality of image processing tools will be described in detail below.

With continued reference to FIG. 1, the ultrasound imaging apparatus 101 may include a probe 126. The probe 126 has elements such as an ultrasonic transducer, a transmitter, a transmit beam former, a detector/SAP electronics, etc., (not shown). The detector/SAP electronics may be used to control the switching of the transducer elements. The detector/SAP electronics may also be used to group the transducer elements into one or more sub-holes. Configurations of the probe 126 will also be described below exemplarily. The probe 126 may be any type of probe, including a linear probe, a curved array probe, a 1.25D array probe, a 1.5D array probe, a 1.75D array probe, or a 2D array probe. According to a preferred embodiment of the present application, the probe 126 may be a probe for volumetric imaging. For example, the probe 126 may be an electronic 4D (E4D) probe. In addition, the probe 126 may also be a mechanical probe, for example, a mechanical 4D probe or a hybrid probe. The probe 126 may be configured to acquire 4D ultrasound data, and the 4D ultrasound data includes information about how the volume changes over time, and may be processed to obtain a volumetric ultrasound image related to the site to be imaged. It can be understood that each volume may include a plurality of 2D images or slices, and accordingly, the controller circuit may select a required 2D image from the volumetric ultrasound images.

The probe 126 may include an ultrasonic transducer. The probe may be configured to acquire ultrasound data or information from tissue to be imaged (e.g., organs such as breasts and the heart, corresponding skin surfaces outside organs, etc.) of a patient. The probe 126 is communicatively connected to the controller circuit by means of the transmitter. The transmitter transmits a signal to the transmit beam former on the basis of acquisition settings received by the controller circuit 102. The acquisition settings may define the amplitude, pulse width, frequency, gain setting, scanning angle, power, time gain compensation (TGC), resolution, and the like of the ultrasonic pulses emitted by the ultrasonic transducer. The ultrasonic transducer emits a pulsed ultrasonic signal into a patient (e.g., the body). The acquisition settings may be defined by a user operating the user interface 142. The signal transmitted by the transmitter, in turn, drives the ultrasonic transducer.

The ultrasonic transducer transmits the pulsed ultrasonic signal to a body (e.g., a patient) or a volume that corresponds to an acquisition setting along one or more scanning planes. The ultrasonic signal may include, for example, one or more reference pulses, one or more push pulses (e.g., shear waves), and/or one or more pulsed wave Doppler pulses. At least a portion of the pulsed ultrasonic signal is backscattered from a tissue to be imaged (e.g., an organ, bone, heart, breast tissue, liver tissue, cardiac tissue, prostate tissue, newborn brain, embryo, abdomen, etc.) to produce an echo. Depending on the depth or movement, the echo is delayed in time and/or frequency, and received by the ultrasonic transducer. The ultrasonic signal may be used for imaging, for producing and/or tracking a shear wave, for measuring changes in location or velocity within the anatomical structure and a compressive displacement difference (e.g., strain) of tissue, and/or for treatment and other applications. For example, the probe 126 may deliver low energy pulses during imaging and tracking, deliver medium and high energy pulses to produce shear waves, and deliver high energy pulses during treatment.

The ultrasonic transducer converts a received echo signal into an electrical signal that can be received by the receiver. The receiver may include one or more amplifiers, analog/digital converters (ADCs), and the like. The receiver may be configured to amplify the received echo signal after appropriate gain compensation, and convert these analog signals received from each transducer element into a digitized signal that is temporally uniformly sampled. The digitized signals representing the received echoes are temporarily stored in the memory 106. The digitized signals correspond to the backscattered waves received by each transducer element at different times. After being digitized, the signal may still retain the amplitude, frequency, and phase information of the backscattered wave.

Optionally, the controller circuit 102 may retrieve the digitized signals stored in the memory 106 for use in a beam former processor. For example, the controller circuit 102 may convert the digitized signal into a baseband signal or compress the digitized signal.

In some embodiments, the controller circuit 102 may further include a beam forming processor. The beam forming processor may include one or more processors. If desired, the beam forming processor may include a central processing unit (CPU), one or more microprocessors, or any other electronic component capable of processing the input data according to specific logic instructions. Additionally or alternatively, the beam forming processor may execute instructions stored on a tangible and non-transitory computer-readable medium (e.g., the memory 106) to perform beam forming computation using any suitable beam forming method, such as adaptive beam forming, synthetic emission focusing, aberration correction, synthetic aperture, clutter suppression, and/or adaptive noise control, etc.

In some embodiments, the controller circuit 102 may further include a radio frequency (RF) processor. The beam forming processor executes beam forming on the digitized signals of the transducer elements, and outputs an RF signal. The RF signal is then provided to the RF processor for processing the RF signal. The RF processor may include one or more processors. If desired, the RF processor may include a central processing unit (CPU), one or more microprocessors, or any other electronic component capable of processing the inputted data according to specific logic instructions. Additionally or alternatively, the RF processor may execute instructions stored on a tangible and non-transitory computer-readable medium (e.g., the memory 106). Optionally, the RF processor may be integrated with and/or be part of the controller circuit 102. For example, operations described as being executed by the RF processor may be configured to be executed by the controller circuit 102.

The RF processor may generate, for a plurality of scanning planes or different scanning modes, different ultrasonic image data types and/or modes, e.g., B-mode, color Doppler (e.g., color blood flow, velocity/power/variance), tissue Doppler (velocity), and Doppler energy, on the basis of a predetermined setting of a first model. For example, the RF processor may generate tissue Doppler data for multiple scanning planes. The RF processor acquires the information (e.g., I/Q, B-mode, color Doppler, tissue Doppler, and Doppler energy information) related to a plurality of data pieces, and stores the data information in the memory 106, where the data information may include time stamp and orientation/rotation information.

Optionally, the RF processor may include a composite demodulator (not shown) for demodulating the RF signal to generate an IQ data pair representing an echo signal. The RF or IQ signal data may be provided directly to the memory 106 so as to be stored (e.g., stored temporarily). If desired, an output of the beam forming processor may be delivered directly to the controller circuit 102.

The controller circuit 102 may be configured to process the acquired ultrasound data (e.g., RF signal data or IQ data pairs), and prepare and/or generate frames of ultrasound image data representing an anatomical structure of interest so as to display the same on the display apparatus 138. The acquired ultrasound data may be processed by the controller circuit 102 in real time during a scanning or treatment process of ultrasound examination when echo signals are received. Additionally or alternatively, the ultrasound data may be temporarily stored in the memory 106 during a scanning process, and processed in a less real-time manner in a live or off-line operation.

The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store medical images, such as a volumetric ultrasound image data set of ultrasound data, where such a volumetric ultrasound image data set is accessed to present two-dimensional and volumetric images. For example, the volumetric ultrasound image data set may be mapped to the corresponding memory 106 and one or more reference planes. Processing of ultrasound data that includes the ultrasound image data set may be based in part on user input, e.g., a user selection received at the user interface 142.

In some embodiments, the ultrasound imaging apparatus 101 may further include a sensing device 108. The sensing device 108 can be used to determine the positional relationship of the probe with respect to the scanned subject in a real-time ultrasound scanning process, thereby determining a real-time ultrasound scanned site of the probe. For the specific configuration and identification of the sensing device, reference may be made to the description of any one of embodiments described below.

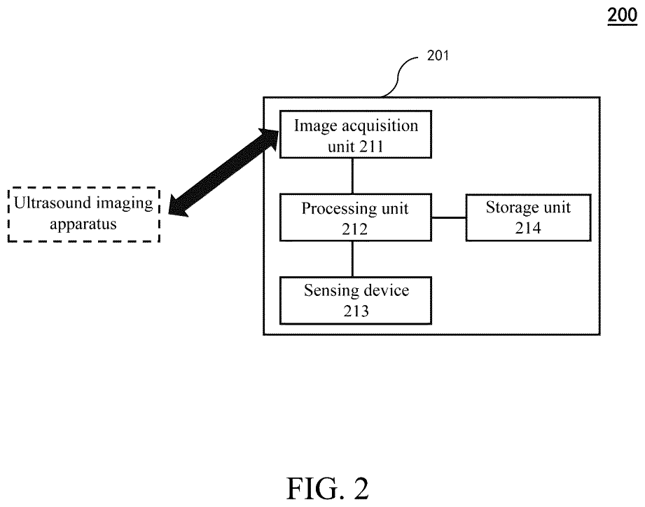

An example of the present application wherein a plurality of image processing tools are integrated in an ultrasound imaging apparatus, and an image processing tool is automatically selected, has been described in the above embodiment. However, the present application is not limited thereto. With reference to FIG. 2, a schematic diagram of an ultrasound system 200 in some other embodiments of the present application is shown.

As shown in FIG. 2, the ultrasound system 200 may include an image processing apparatus 201. The image processing apparatus 201 may include an image acquisition unit 211 and a processing unit 212. The image acquisition unit 211 is used to acquire the ultrasound image, and the processing unit 212 is configured to perform image processing using the real-time ultrasound image acquired by the image acquisition unit 211. The specific processing manner will be described in detail below, and FIG. 2 describes a connection relationship and principle of each component by way of example.

Optionally, the image processing apparatus 201 may include a sensing device 213. The sensing device 213 may sense the relative position of the probe (not shown) with respect to the scanned subject in the real-time ultrasound scanning process, and send same to the processing unit 212. Thus, it is advantageous for the processing unit 212 to select a corresponding image processing tool to process the real-time ultrasound image. The specific configuration of the sensing device will be described in detail below. It should be noted that FIG. 2 shows a situation in which the sensing device is integrated into the image processing apparatus 201. In some examples, the image processing apparatus 201 may not include the sensing device 213. For example, the sensing device 213 may be configured to be integrated on the ultrasound imaging apparatus 202. Alternatively, the sensing device 213 may be a separate external device, and the image processing apparatus 201 only integrates the processing algorithm of the image acquired by the sensing device 213.

It can be understood that the ultrasound system 200 in the above embodiment, particularly in comparison with the ultrasound imaging apparatus 101, may be considered as an external device that is independent of the ultrasound imaging apparatus, and is specifically used for ultrasound image processing. The system itself is provided with an independent processing unit 212 and various algorithms used for image processing. In ultrasound imaging, it is difficult to upgrade the software configuration of an ultrasound imaging apparatus because of considerations such as security. An initially used ultrasound imaging apparatus is not provided with functions such as a plurality of different image processing tools, and it is difficult to install new functions later. In the example of FIG. 2, the image processing apparatus 201 in the form of an external device has great flexibility. It is easier to iterate, and further, it can integrate software products from different manufacturers and thus often has powerful functionality. However, operating the external image processing apparatus 201 during a real-time ultrasound scanning process also poses a number of difficulties compared with an integrated ultrasound imaging apparatus. For example, when the two hands of the user operate the probe and the user interface of the ultrasound imaging apparatus, it is difficult to operate the image processing apparatus 201 to select a function. This results in great inconvenience in use. Accordingly, applying the image processing method described in any one of the embodiments of the present application to the image processing apparatus 201 will significantly improve the efficiency of real-time ultrasound imaging and save time for the user, because it can largely eliminate excess operations of the image processing apparatus 201 by the user.

In some embodiments, the image processing apparatus 201 further includes a storage unit 214. The storage unit 214 may be used to store various algorithms for the processing unit 212. For example, it may store various image processing tools, an automatic selection algorithm for the image processing tools, etc. As described above, these algorithms may be from different manufacturers. Also, in an example of the present application, there is no need to consider compatibility between algorithms of different manufacturers. This is because the image processing apparatus 201 can automatically select algorithms (e.g., image processing tools from different manufacturers). After determining an image processing tool that needs to be used, the image processing tool from a certain manufacturer is independently operated, and thus there is no compatibility problem.

In some embodiments, the ultrasound system 200 may be considered as an independent device including only the image processing apparatus 201. In another example, the ultrasound system 200 may further include an ultrasound imaging apparatus 202. The image acquisition unit 211 is connected to the ultrasound imaging apparatus 202, so as to acquire the ultrasound image. Although not shown in FIG. 2, it can be understood that the ultrasound imaging apparatus 202 may include a probe, a processor, and other components that perform ultrasound imaging. The probe is used to acquire an ultrasonic echo signal of a currently scanned site, and the processor is used to process the ultrasonic echo signal to generate the ultrasound image. To this end, in real-time ultrasound scanning, the ultrasound imaging apparatus 202 of the ultrasound system 200 is used for real-time imaging, and the imaging results are sent to the image processing apparatus 201 for processing. Moreover, the built-in algorithm of the image processing apparatus 201 can automatically select an appropriate image processing tool, thereby eliminating a manual operation workflow.

It should be noted that for the specific configuration of the ultrasound imaging apparatus 202 shown in FIG. 2, for example, the probe and the processor, reference may be made to any one of the embodiments of the ultrasound imaging apparatus 101 corresponding to FIG. 1 of the present application. For another example, a communication circuit may be used for wired or wireless data transmission and the like with the image acquisition unit 211. The two mainly differ in that, in the example shown in FIG. 2, an algorithm related to a plurality of ultrasound image processing tools and an algorithm for automatic selection of said tools are integrated at the image processing apparatus 201.

An exemplary description of the ultrasound image processing method will be provided below. The method is suitable for automatically selecting, from a plurality of image processing tools, an appropriate image processing tool for processing the ultrasound image in real-time ultrasound scanning. It can be understood that the image processing method described in the following embodiments is suitable for the system/apparatus described in any one of the embodiments of the present application, e.g., the ultrasound system 200 and/or the ultrasound imaging apparatus 101. The processor of the above system/apparatus may be used to perform the image processing method described in the embodiments of the present application.

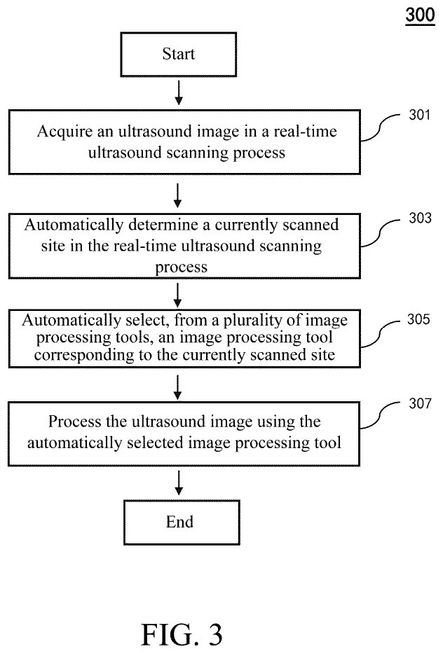

With reference to FIG. 3, a flowchart of an ultrasound image processing method 300 in some embodiments of the present application is shown.

In step 301, an ultrasound image in a real-time ultrasound scanning process is acquired, the ultrasound image being generated by processing an ultrasonic echo signal of a currently scanned site acquired by a probe.

In step 303, the currently scanned site is automatically determined in the real-time ultrasound scanning process.

In step 305, an image processing tool corresponding to the currently scanned site is automatically selected from a plurality of image processing tools, wherein the plurality of image processing tools correspond to a plurality of scanned sites of a scanned subject, respectively.

In step 307, the ultrasound image is processed using the automatically selected image processing tool.

The above configuration can help a user (e.g., an ultrasound technician) improve scanning efficiency in a real-time ultrasound scanning process. Specifically, unlike a mode in which scanning and image processing are separated, in real-time ultrasound scanning, both hands of the user need to continuously operate the ultrasound imaging apparatus, and selecting an additional ultrasound image processing tool requires a large amount of time and interrupts the continuity of the ultrasound scanning. In the above method of the present application, the scanned site is automatically determined in the real-time ultrasound scanning process, and the image processing tool corresponding to the scanned site is automatically selected according to the scanned site, which both ensures that the selected image processing tool is suitable for the current scan, and does not require the user to perform additional operations. It is particularly suitable for real-time ultrasound scanning.

In the above embodiment, the generation of the ultrasound image in the real-time ultrasound scanning process may be implemented by any ultrasound imaging apparatus of the present application or the prior art. Said image is acquired by the processor of the ultrasound imaging apparatus itself, or may be acquired by processing of the above image processing apparatus.

Further, automatically determining the currently scanned site may be implemented in different manners. In one example, it may be implemented by means of image identification. For a specific algorithm for image identification, reference may be made to the description of any one of the embodiments herein. For example, it may be implemented by means of an artificial intelligence model. For example, image identification may be performed on a real-time ultrasound image while the ultrasound image is being acquired, and it may be determined which organ/tissue section the ultrasound image corresponds to, thereby determining the currently scanned site.

However, the inventors have realized that it may be difficult to determine a scanned site by image identification. One of the main reasons is that the resolution of the ultrasound image is limited, and greatly affected by the user's operation of the probe. It is difficult to accurately identify scanned sites for many ultrasound images. It is usually necessary to acquire standard or representative sections in order to be able to perform accurate automatic identification. This may add an extra burden. For at least the above reasons, more accurate solutions are provided in the embodiments of the present application.

In some embodiments, automatically determining the currently scanned site may include: determining a relative position of the probe with respect to the scanned subject using a sensing device; and determining the currently scanned site on the basis of the relative position.

The inventors have realized that in the real-time ultrasound scanning process, the relative position of the probe and the scanned subject can effectively assist in determining the scanned site of the currently scanned subject. For example, if a probe is resting on a subject's breast, it may be considered to correspond to a breast scan. Alternatively, if the probe is resting on the skin surface above the liver, it may be considered that the liver is being scanned. Compared with simple image identification, sensing the relative positional relationship between the probe and the scanned subject does not require a demanding scanning technique, and does not add an extra burden on the user.

It should be noted that in some other embodiments, the identification manner for determining the relative position of the probe with respect to the scanned subject may also be combined with other identification manners. For example, this identification manner may be used together with the manner of image identification described above. The manner of identifying the relative position of the probe and the scanned subject can be assisted by image identification of real-time ultrasound images of the scanned sites. This may be advantageous in situations where scanned sites are relatively close. For example, a breast scan and a heart scan may have overlapping positions. Although the relative position of the probe and the scanned subject may be detected by the sensing device to distinguish between them (e.g., breast and heart scanning procedures and motions may be significantly different, or different probes may need to be used, resulting in different sensing signals), the efficiency may be reduced. In this case, the efficiency of identifying the scanned site can be improved by combining the two methods.

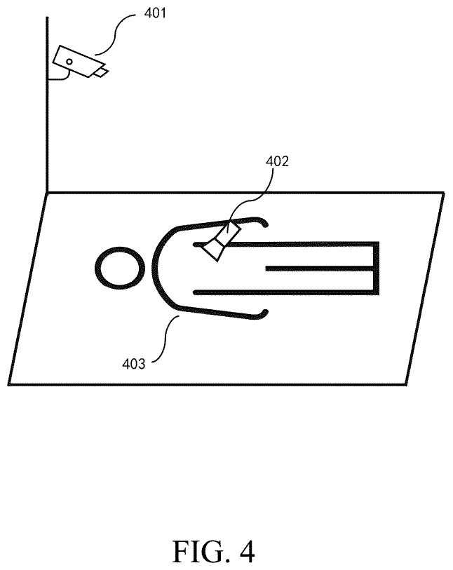

An exemplary description of the configuration of the sensing device will be provided below. With reference to FIG. 4, a schematic diagram 400 of using a sensing device to determine a relative position of a probe 402 with respect to a scanned subject 403 in some embodiments of the present application is shown.

In FIG. 4, the sensing device may include a camera 401. The camera 401 can acquire an environment image including the probe 402 and the scanned subject 403. Further, the relative position of the probe with respect to the scanned subject is determined by identifying the probe 402 and the scanned subject 403 in the environment image.

Such a configuration enables the relative position between the probe 402 and the scanned subject 403 to be determined quickly and accurately. Thus, the currently scanned site can be determined according to the relative position.

In this embodiment, the camera 401 may be disposed at a high position such as a wall, thereby obtaining a global environment image for further analysis. The type of the camera 401 may be a 2D camera, a 3D camera or an infrared camera, and may have a video recording function. Also, the identification and analysis algorithm of the environment image may be any technique in the prior art, and details will not be described again here.

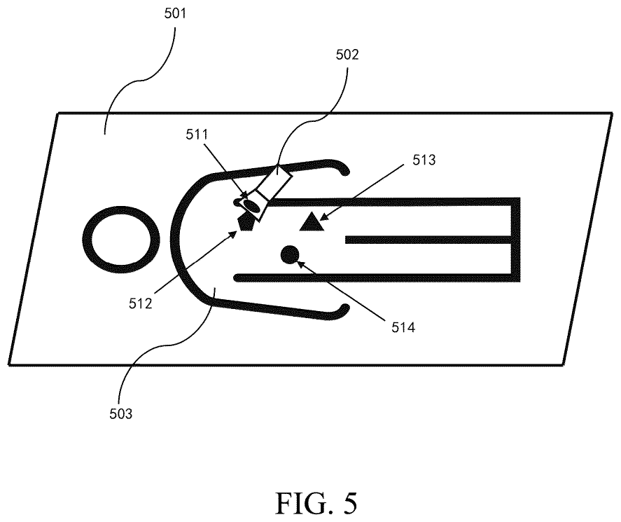

In addition, the sensing device may also be another means. With reference to FIG. 5, a schematic diagram 500 of using a sensing device to determine a relative position of a probe 502 with respect to a scanned subject 503 in some other embodiments of the present application is shown.

As shown in FIG. 5, the sensing device may include a first positioning apparatus 511 and a plurality of second positioning apparatuses 512-514. The first positioning apparatus 511 is disposed in the probe 502, and the plurality of second positioning apparatuses 512-514 are disposed corresponding to different sites of the scanned subject 503, respectively. Furthermore, the first positioning apparatus 511 approaches one of the plurality of second positioning apparatuses 512-514 and generates a sensing signal, and the relative position of the probe 502 with respect to the scanned subject 503 is determined on the basis of the sensing signal. For example, as shown in FIG. 5, a sensing signal is generated when the first positioning apparatus 511 and the second positioning apparatus 512 are close to each other, and at this time, it can be determined that the probe 502 is at a position near the second positioning apparatus 512. Moreover, if the second positioning apparatus 512 is disposed on a left breast, it can be determined that the probe 502 is scanning the left breast of the scanned subject 503.

The first and second positioning apparatuses 511-514 may be disposed in various manners. For example, it may be any technique in the art by which a sensor signal is generated by means of approaching each other (for example, a change in an electric or magnetic signal due to the approach), such as an electrical/magnetic sensor. The present application does not make a limitation. In addition, in some examples, the sensing signal may be sent by the first positioning apparatus 511 on the probe 502 to a processor of an image processing apparatus/system for analysis and determination. For example, the first positioning apparatus 511 may be integrated into a transducer end of the probe 502. In a further embodiment, the described signal may be sent by the second positioning apparatus 512. It may be sent in a wired or wireless manner. In addition, regarding the installation position of the second positioning apparatus 512, it may be installed on an ultrasound scanning table 501, or may be attached to the body surface of the scanned subject 503 in advance.

As described above, the sensing device in the embodiments of FIGS. 4 and 5 of the present application may be integrated in the ultrasound imaging apparatus/ultrasound system/image processing apparatus described in any one of the embodiments of the present application (for example, it may be the sensing device 108, 213), or may be an external device. In addition, other components such as the ultrasound imaging apparatus are not shown in FIGS. 4 and 5, for ease of understanding, but for said other components, reference may also be made to the description of any one of the embodiments herein.

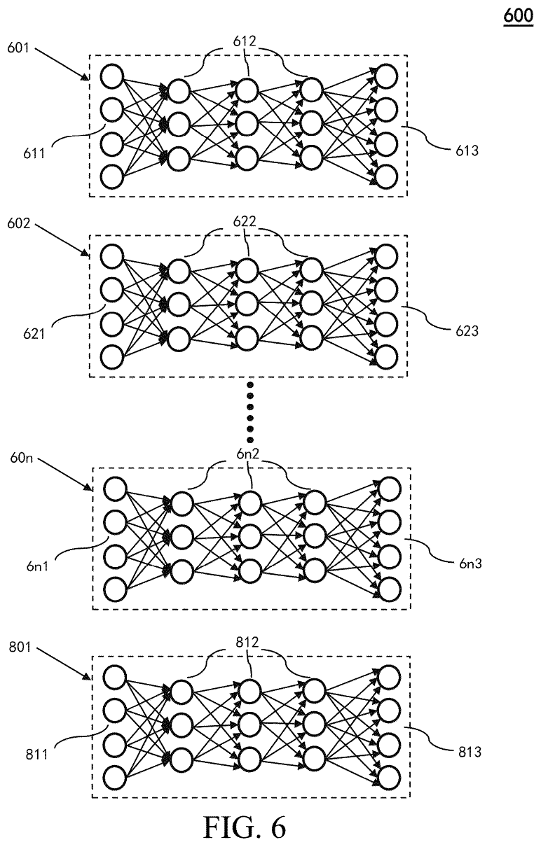

After automatically determining the scanned site, an image processing tool corresponding to the currently scanned site may be automatically selected from the plurality of image processing tools. For the configuration of the plurality of image processing tools, reference may be made to the description of FIG. 6 of the present application. FIG. 6 shows a schematic diagram of a plurality of image processing tools 600 in some embodiments of the present application.

In some examples, the plurality of image processing tools 600 may include a plurality of artificial intelligence image processing tools 601-60n, the plurality of artificial intelligence image processing tools 601-60n being obtained by training for the different sites, respectively. For example, the artificial intelligence image processing tools may be obtained by training using any existing artificial intelligence technique, such as a deep learning technique. The artificial intelligence image processing tools will be described below by way of example, but it should be noted that the present application does not provide an exclusive definition, and any artificial intelligence techniques in the art is allowed.

In some embodiments, an artificial neural network using deep learning technology is trained on the basis of a Residual Network (ResNet), a Visual Geometry Group Network (VGGNet) or another well-known model. Since the number of processing layers in the ResNet can be set large (as large as 1000 or more), classification (for example, determination of artifact type) based on this network structure can achieve a better effect. In addition, it is easier for the ResNet to optimize the learning network based on more training data.

An artificial intelligence image processing tool 601 will be described as an example. A learning network thereof includes an input layer 611, a processing layer (also referred to as a hidden layer) 612, and an output layer 613. In some embodiments, as shown in FIG. 6, the processing layer 612 includes a first convolutional layer, a first pooling layer, and a fully-connected layer (that is, three layers from left to right in the processing layer 612). The first convolutional layer is used to perform convolution of each input parameter, to obtain a feature map of the first convolutional layer. The first pooling layer pools (down-samples) the feature map of the first convolutional layer, to compress the feature map of the first convolutional layer and extract main features thereof, so as to obtain a feature map of the first pooling layer. The fully-connected layer may output a determination result on the basis of the feature map of the first pooling layer.

Although FIG. 6 shows the example of only one convolutional layer, in other examples, there may be any number of convolutional layers, and the number of the convolutional layers may be appropriately adjusted according to the size of input data in the learning network, etc. For example, a second convolutional layer and a second pooling layer (not shown) may be further included between the first pooling layer and the fully-connected layer, or a second convolutional layer and a second pooling layer, as well as a third convolutional layer and a third pooling layer (not shown), and so on may be further included between the first pooling layer and the fully-connected layer.

Although FIG. 6 only shows that the convolutional layer is connected to the input layer, the pooling layer is connected to the convolutional layer, and the fully-connected layer is connected to the pooling layer, in other examples, any number of processing layers of any type may be provided between any two of the aforementioned layers. For example, a normalization layer may be provided between the convolutional layer and the input layer to normalize the input parameters, or an activation layer may be provided between the fully-connected layer and the pooling layer to perform nonlinear mapping on a feature map of the pooling layer using a Rectified Linear Unit (ReLU) activation function.

In some embodiments, each layer includes several neurons (i.e., circle patterns in the figure), and the number of neurons in each layer may be configured to be the same or different according to need. A sample data set (a known input) and an anesthetic parameter (an expected output) are input into the learning network, a number of processing layers in the learning network and a number of neurons in each of the processing layers are configured, and the weight and/or bias of the learning network is estimated (or regulated or calibrated), so as to identify a mathematical relationship between the known input and the expected output and/or identify and characterize a mathematical relationship between the input and output of each layer. The learning process generally uses (some) input data, and creates a network output for the input data; then, the created network output corresponding to the known input is compared with the expected output of the data set, the difference thereof being a loss function; and the loss function is used to iteratively update network parameters (weight and/or bias) to continuously decrease the loss function, so as to train a neural network model having higher accuracy. In some embodiments, many functions can be used as the loss function, including, but not limited to, mean squared error (mean suqared), cross entropy error, and the like.

In one embodiment, although the configuration of the learning network of the artificial intelligence image processing tool 601 is guided by dimensions such as priori knowledge, input, and output of an estimation problem, the learning itself is regarded as a “black box,” and implements optimal approximation of required output data mainly depending on or exclusively according to input data. In various alternative implementations, clear meaning may be assigned to some data representations in the learning network using some aspects and/or features of data, imaging geometry, a reconstruction algorithm, or the like. This helps to accelerate training. This creates an opportunity to separately train (or pre-train) or define some layers in the learning network.

It is understood that a deep learning method is characterized by the use of one or more network architectures to extract or simulate data of interest. The deep learning method may be implemented using one or a plurality of processing layers (for example, an input layer, an output layer, a convolutional layer, a normalization layer, a sampling layer, or the like, where processing layers of different numbers and functions may exist according to different deep learning network models), where the configuration and number of the layers allows a deep learning network to process complex information extraction and modeling tasks. Specific parameters (also referred to as “weight” or “bias”) of the network are usually estimated by means of a so-called learning process (or training process). The learned or trained parameters usually result in (or output) a network corresponding to layers of different levels, so that extraction or simulation of different aspects of initial data or the output of a previous layer usually may represent the hierarchical structure or concatenation of layers. Processing may be performed layer by layer, that is, “simple” features may be correspondingly extracted from input data for an earlier or higher-level layer, and then these simple features are combined into a layer exhibiting features of higher complexity. In practice, each layer (or more specifically, each “neuron” in each layer) may process input data as output data for representation by using one or a plurality of linear and/or non-linear transformations (so-called activation functions). The number of the plurality of “neurons” may be constant among the plurality of layers or may vary from layer to layer.

As discussed herein, as part of initial training of a deep learning process for solving a specific problem, a training data set includes a known input value and an expected (target) output value (e.g., a determination result) finally outputted in the deep learning process. In this manner, a deep learning algorithm can (in a supervised or guided manner or an unsupervised or unguided manner) process the training data set until a mathematical relationship between a known input and an expected output is identified and/or a mathematical relationship between the input and output of each layer is identified and represented. In the learning process, (a part of) input data is usually used, and a network output is created for the input data. Afterwards, the created network output is compared with the expected output of the data set, and then the difference between the created and expected outputs is used to iteratively update network parameters (weight and/or bias). A stochastic gradient descent (SGD) method may usually be used to update network parameters. However, those skilled in the art should understand that other methods known in the art may also be used to update network parameters. Similarly, a separate validation data set may be used to validate a trained learning network, where both a known input and an expected output are known. The known input is provided to the trained learning network so that a network output can be obtained, and then the network output is compared with the (known) expected output to validate prior training and/or prevent excessive training.

When the learning network is created or trained, only real-time ultrasound images to be processed need to be input into the artificial intelligence image processing tool 601, and the learning network thereof can automatically process the ultrasound images. Similarly, for the construction and training of the artificial intelligence image processing tools 602-60n, reference may be made to the description of the above embodiments of the present application. For example, the learning network of the artificial intelligence image processing tool 602 includes an input layer 621, a processing layer (also referred to as a hidden layer) 622, and an output layer 623. For another example, the learning network of the artificial intelligence image processing tool 60n includes an input layer 6n1, a processing layer (also referred to as a hidden layer) 6n2, and an output layer 6n3. However, although the described training process is to more clearly describe the artificial intelligence image processing tool of the present application, the artificial intelligence image processing tool of the present application is not limited to being obtained by the described training method.

In one example, the plurality of artificial intelligence image processing tools 601-60n are obtained by training for the different sites, respectively. Such a configuration ensures that different artificial intelligence image processing tools have a higher degree of accuracy and correlation for their corresponding sites. In a non-limiting example, the plurality of artificial intelligence image processing tools 601-60n may be obtained by training for different tissues/sites to be scanned, respectively, such as heart, breast, liver, kidney, carotid artery, skeletal muscle, fetus, etc. The foregoing will not be further enumerated.

It can be understood that the ultrasound images may be processed in various manners. In some embodiments, processing the ultrasound image includes performing at least one of quality optimization, image identification, anatomical feature identification, lesion identification, image segmentation and image measurement on the ultrasound image; and on the basis of different scanned sites, at least some of the image processing tools perform different processing on the ultrasound image. The inventors have realized that different scanned sites may require different manners of processing the ultrasound image. For example, for a lesion screening process, a more urgently needed image processing manner is lesion identification. For fetal examination, identification of anatomical features and image measurement (e.g., length, area, etc. of specific physiological features of the fetus) may be more desirable. In an embodiment of the present application, at least some of the plurality of different image processing tools 601-60n are provided with different image processing functions, thereby better processing ultrasound images of different sites. This can improve the working efficiency and the degree of satisfaction of the automatic ultrasound image processing tool.

In one example, the plurality of image processing tools 600 described above are integrated into an ultrasound imaging apparatus as described in any one of the embodiments of the present application, which can increase the degree of integration of the apparatus.

In a further example, the plurality of image processing tools 600 described above are integrated in an ultrasound system as described in any one of the embodiments of the present application, e.g., in an image processing apparatus of the ultrasound system. The image processing apparatus is provided independently of the ultrasound imaging apparatus. It receives the real-time ultrasound images of the ultrasound imaging apparatus by means of a data transmission tool and processes the real-time ultrasound images through an internal algorithm (such as algorithms of a plurality of image processing tools 600). Such a configuration makes it easier to update the image processing tool and meet user needs in a timely manner. For example, in one scenario, at least some of the plurality of image processing tools 600 are from different providers (e.g., tool developers). The image processing apparatus provides a versatile platform for integration of different tools, and can automatically select one of the plurality of different image processing tools 600 described above by the example of the present application without considering compatibility between the different tools and other problems. When image processing tools need to be added or updated, it is also easier for independent image processing tools 600 to update iterations. In addition, the processed ultrasound image may be displayed either directly by the image processing apparatus of the ultrasound system or by the ultrasound imaging apparatus.

It should be noted that FIG. 6 shows a case in which the plurality of image processing tools are artificial intelligence image processing tools. Non-artificial intelligence image processing tools, such as tools generated by classical machine learning models or other algorithms, are also allowed in the embodiments of the present application.

The inventors have realized that in some examples, there may be situations in which an image processing tool cannot be selected. Although processing may be performed by manual intervention at that time, this inevitably affects operation efficiency of the user. For at least this reason, improvements are further provided in some embodiments of the present application.

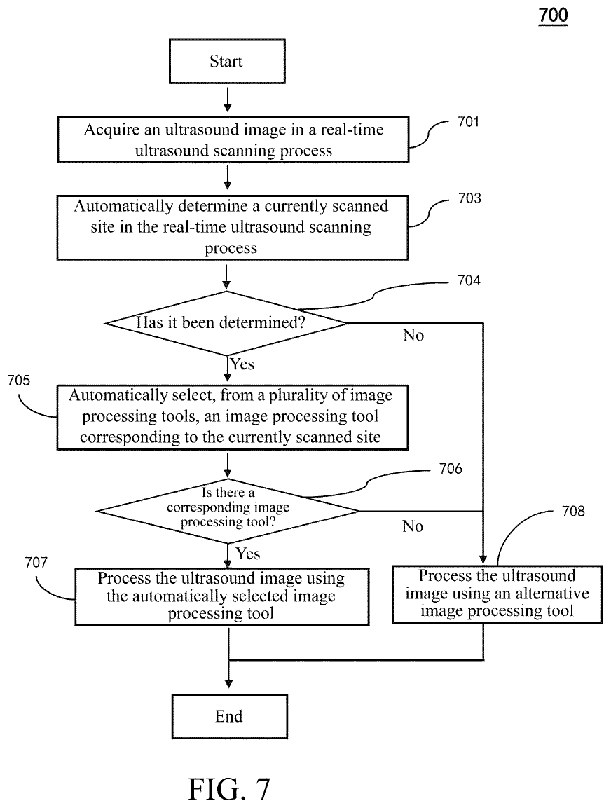

First, with reference to FIG. 7, a flowchart of an ultrasound image processing method 700 in some other embodiments of the present application is shown.

In step 701, an ultrasound image in a real-time ultrasound scanning process is acquired, the ultrasound image being generated by processing an ultrasonic echo signal of a currently scanned site acquired by a probe.

In step 703, the currently scanned site is automatically determined in the real-time ultrasound scanning process.

In step 704, a determination is made as to whether the currently scanned site has been automatically determined; if so, step 705 is performed, and if not, step 708 is performed.

In step 705, an image processing tool corresponding to the currently scanned site is automatically selected from a plurality of image processing tools, wherein the plurality of image processing tools correspond to a plurality of scanned sites of a scanned subject, respectively.

In step 706, a determination is made as to whether there is an image processing tool corresponding to the currently scanned site; if so, step 707 is performed, and if not, step 708 is performed.

In step 707, the ultrasound image is processed with the automatically selected image processing tool.

In step 708, the ultrasound image is processed with an alternative image processing tool.

For specific implementation of steps 701, 703, 705 and 707, reference may be made to FIG. 3 of the present application and the description of the corresponding embodiment, and details will not be described again here. In addition to the above, the method 700 of the present application improves the fault tolerance rate of the algorithmic tool. That is, in this embodiment, in response to the currently scanned site not being determined, or in response to an inability to automatically select, from the plurality of image processing tools, an image processing tool corresponding to the currently scanned site, the processor can process the ultrasound image using an alternative image processing tool. Such a configuration makes it possible to compensate for problems caused by erroneous identification of the scanned site or insufficient coverage of the image processing tool, thereby further improving the image processing efficiency of the user.

Regarding the alternative image processing tool, please refer back to FIG. 6. One example of an alternative image processing tool 801 is shown. The alternative image processing tool 801 may be an artificial intelligence image processing tool, which may be configured with reference to other processing tools shown in FIG. 6. For example, it has a learning network, and the learning network may include an input layer 821, a processing layer (also referred to as a hidden layer) 822, and an output layer 823. The main difference may be that the processing range of the alternative image processing tool 801 can have wider versatility. This can be achieved during model training. For example, when training a model, a plurality of ultrasound images of different sites are selected for hybrid training, thereby increasing the versatility of the alternative image processing tool 801. In addition, the type of the output layer 823 may be defined. For example, a general type of the output layer 823 is selected, e.g., a type of image processing required for most ultrasound scans, such as improved ultrasound image quality, identification of anatomical structures, etc. That is, the so-called alternative image processing tool 801 is capable of processing a plurality of different sites. This will provide a compromise remedy for image processing errors. That is, the success rate of image processing is improved at the expense of the targetedness of the image processing, to a certain extent.

Some embodiments of the present application further provide a non-transitory computer-readable medium, wherein the non-transitory computer-readable medium has a computer program stored thereon, the computer program has at least one code segment, and the at least one code segment is executable by a machine so as to enable the machine to perform the steps of the method described in any one of the above embodiments of the present application.

Some embodiments of the present application provide an ultrasound imaging apparatus, including a probe and a processor. The probe is used to acquire an ultrasonic echo signal of a currently scanned site. The processor is configured to perform the steps of the method described in any one of the above embodiments of the present application. For a specific configuration of the ultrasound imaging apparatus described above, for example, a configuration of the probe and the processor, and a configuration of other components, reference may also be made to the description of any one of the embodiments described above, and details will not be described again.

Some embodiments of the present application provide an ultrasound system, including an image processing apparatus. The image processing apparatus comprises an image acquisition unit and a processing unit. The image acquisition unit is used to acquire the ultrasound image. The processor is configured to perform the steps of the method described in any one of the above embodiments of the present application. For specific configurations of the ultrasound system and the image processing apparatus, reference may be made to the description of any one of the above embodiments.

Correspondingly, the present disclosure may be implemented by means of hardware, software, or a combination of hardware and software. The present disclosure may be implemented in at least one computer system in a centralized manner, or implemented in a distributed manner; and in the distributed manner, different elements are distributed on a plurality of interconnected computer systems. Any type of computer system or other apparatus suitable for implementing the methods described herein is considered to be appropriate.

Various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of the following: a) conversion to another language, code, or symbol; and b) replication in different material forms.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. An ultrasound image processing method, comprising:
acquiring an ultrasound image in a real-time ultrasound scanning process, the ultrasound image being generated by processing an ultrasonic echo signal of a currently scanned site acquired by a probe;
automatically determining the currently scanned site in the real-time ultrasound scanning process;
automatically selecting, from a plurality of image processing tools, an image processing tool corresponding to the currently scanned site, wherein the plurality of image processing tools correspond to a plurality of scanned sites of a scanned subject, respectively; and
processing the ultrasound image using the automatically selected image processing tool.

2. The method according to claim 1, wherein the automatic determination of the currently scanned site comprises:
determining a relative position of the probe with respect to the scanned subject using a sensing device; and
determining the currently scanned site on the basis of the relative position.

3. The method according to claim 2, wherein:
the sensing device comprises a camera, the camera acquiring an environment image comprising the probe and the scanned subject; and
the relative position of the probe with respect to the scanned subject is determined by means of identifying the probe and the scanned subject in the environment image.

4. The method according to claim 2, wherein:
the sensing device comprises a first positioning apparatus and a plurality of second positioning apparatuses, the first positioning apparatus being disposed on the probe, and the plurality of second positioning apparatuses being disposed corresponding to different sites of the scanned subject, respectively; and
the first positioning apparatus approaches one of the plurality of second positioning apparatuses and generates a sensing signal, and the relative position of the probe with respect to the scanned subject is determined on the basis of the sensing signal.

5. The method according to claim 1, wherein:
the plurality of image processing tools comprise a plurality of artificial intelligence image processing tools, and the plurality of artificial intelligence image processing tools are obtained by training for the different sites, respectively.

6. The method according to claim 1, wherein:
processing the ultrasound image comprises performing at least one of quality optimization, image identification, anatomical feature identification, lesion identification, image segmentation and image measurement on the ultrasound image; and on the basis of different scanned sites, at least some of the image processing tools perform different processing on the ultrasound image.

7. The method according to claim 1, further comprising:
in response to the currently scanned site not being determined, or in response to an inability to automatically select, from the plurality of image processing tools, the image processing tool corresponding to the currently scanned site, processing the ultrasound image using an alternative image processing tool.

8. An ultrasound imaging apparatus, comprising:
an image processing apparatus, the image processing apparatus comprising: a probe used to acquire an ultrasound image;
a memory storing instructions; and
a processor configured to execute the instructions to:
acquire an ultrasound image in a real-time ultrasound scanning process, the ultrasound image being generated by processing an ultrasonic echo signal of a currently scanned site acquired by a probe;
automatically determine the currently scanned site in the real-time ultrasound scanning process;
automatically select, from a plurality of image processing tools, an image processing tool corresponding to the currently scanned site, wherein the plurality of image processing tools correspond to a plurality of scanned sites of a scanned subject, respectively; and
process the ultrasound image using the automatically selected image processing tool.

9. The ultrasound imaging apparatus according to claim 8, further comprising:
a sensing device sensing a relative position of the probe with respect to a scanned subject, and sending same to the processor.

10. The method according to claim 8, wherein the automatic determination of the currently scanned site comprises:
determining a relative position of the probe with respect to the scanned subject using a sensing device; and
determining the currently scanned site on the basis of the relative position.

11. The method according to claim 10, wherein:
the sensing device comprises a camera, the camera acquiring an environment image comprising the probe and the scanned subject; and
the relative position of the probe with respect to the scanned subject is determined by means of identifying the probe and the scanned subject in the environment image.

12. The method according to claim 10, wherein:
the sensing device comprises a first positioning apparatus and a plurality of second positioning apparatuses, the first positioning apparatus being disposed on the probe, and the plurality of second positioning apparatuses being disposed corresponding to different sites of the scanned subject, respectively; and
the first positioning apparatus approaches one of the plurality of second positioning apparatuses and generates a sensing signal, and the relative position of the probe with respect to the scanned subject is determined on the basis of the sensing signal.

13. The method according to claim 8, wherein:
the plurality of image processing tools comprise a plurality of artificial intelligence image processing tools, and the plurality of artificial intelligence image processing tools are obtained by training for the different sites, respectively.

14. The method according to claim 8, wherein:
processing the ultrasound image comprises performing at least one of quality optimization, image identification, anatomical feature identification, lesion identification, image segmentation and image measurement on the ultrasound image; and on the basis of different scanned sites, at least some of the image processing tools perform different processing on the ultrasound image.

15. The method according to claim 8, wherein the processor is further configured to execute the instructions to:
in response to the currently scanned site not being determined, or in response to an inability to automatically select, from the plurality of image processing tools, the image processing tool corresponding to the currently scanned site, processing the ultrasound image using an alternative image processing tool.

16. A non-transitory, computer readable medium storing instructions that, when executed by a processor, cause the processor to:
acquire an ultrasound image in a real-time ultrasound scanning process, the ultrasound image being generated by processing an ultrasonic echo signal of a currently scanned site acquired by a probe;
automatically determine the currently scanned site in the real-time ultrasound scanning process;
automatically select, from a plurality of image processing tools, an image processing tool corresponding to the currently scanned site, wherein the plurality of image processing tools correspond to a plurality of scanned sites of a scanned subject, respectively; and
process the ultrasound image using the automatically selected image processing tool.

17. The method according to claim 16, wherein the automatic determination of the currently scanned site comprises:
determining a relative position of the probe with respect to the scanned subject using a sensing device; and
determining the currently scanned site on the basis of the relative position.

18. The method according to claim 16, wherein:
the plurality of image processing tools comprise a plurality of artificial intelligence image processing tools, and the plurality of artificial intelligence image processing tools are obtained by training for the different sites, respectively.

19. The method according to claim 16, wherein:
processing the ultrasound image comprises performing at least one of quality optimization, image identification, anatomical feature identification, lesion identification, image segmentation and image measurement on the ultrasound image; and on the basis of different scanned sites, at least some of the image processing tools perform different processing on the ultrasound image.

20. The method according to claim 16, wherein the processor is further configured to execute the instructions to:
in response to the currently scanned site not being determined, or in response to an inability to automatically select, from the plurality of image processing tools, the image processing tool corresponding to the currently scanned site, processing the ultrasound image using an alternative image processing tool.
